(19) United States
(12) Patent Application Publication
(10) Pub. No.: US 2022/0397492 A1
(43) Pub. Date: Dec. 15, 2022

(54) CRUDE CONTAINER WITH INTEGRATED WATER CUT METER

(71) Applicant: Welker, Inc., Sugar Land, TX (US)

(72) Inventors: James T. Klentzman, Wharton, TX (US); Fabio Arenas, The Woodlands, TX (US)

(21) Appl. No.: 17/806,605

(22) Filed: Jun. 13, 2022

Related U.S. Application Data
(60) Provisional application No. 63/210,141, filed on Jun. 14, 2021.

(51) Int. Cl. G01N 1/14; G01N 33/28; G01N 21/78

(52) U.S. Cl. CPC G01N 1/14; G01N 33/2823; G01N 33/2847; G01N 21/78

(57) ABSTRACT

A system is provided for testing and sampling of crude oil that includes a crude oil sampler system with a circulation loop and a water cut meter incorporated into the circulation loop. The system provides for a water cut meter positioned downstream of a sample withdrawal point. As the sample of crude oil passes through the circulation loop, the water cut meter can be used to determine the percentage of water cut of the sample. After passing through the circulation loop and determination of the water cut, a secondary sub-sample of the crude oil may be drawn off from a draw-off valve. The secondary sub-sample may then be sent to a laboratory or testing center for further testing or verification of the initial water cut.

### CROSS-REFERENCE TO RELATED APPLICATION

This application claims the benefit of the filing date of U.S. Provisional Application No. 63/210,141 filed Jun. 14, 2021, entitled CRUDE CONTAINER WITH INTEGRATED WATER CUT METER, which is hereby incorporated by reference as if fully set forth herein.

### FIELD OF THE INVENTION

The present invention is generally directed to crude oil sampling and testing.

### BACKGROUND OF THE INVENTION

It is well known that the purity and composition of crude oil is critical in its effectiveness of being used as fuel, plastics, and other uses. Crude oil that contains unacceptable levels of impurities, such as water or other undesirable elements, is unable to be used for industrial and other purposes. It is also well known that crude oil is bought and sold based on several quality aspects of the particular crude oil in question. Examples would be hydrogen sulfide (H₂S) content, viscosity, sediment, and similar contaminants. Water produced from the formation or water from any other source is a major consideration in the sale or purchase of crude oil. In the field, the water content at high percentages is often dealt with by using oil and water monitors and diverter valves that prevent “bad” oil from crossing the LACT (Lease Automatic Custody Transfer) skid. These systems are typically on two inch (2″) to six inch (6″) piping systems on the LACT skid.

In the field, sampling systems are also installed for determining the quality of the crude that has successfully passed the LACT unit but still includes contaminant content. These systems take a sample over a predetermined period of time. Samples are taken proportional to flow during that time frame. Oftentimes, operators or others collecting the crude oil sample use a container (typically two to ten gallons in volume) that collects the sample of crude oil that is extracted from the main pipeline. After drawing off, the collected crude oil sits for a time in the container so that it can stratify for initial separation. After initial separation, the collected crude oil is shipped to a laboratory or testing center for continued testing, typically in the form of a remixture placed in a centrifuge machine, water by distillation, Karl Fischer titration, or other known or foreseeable methods. Once the testing is completed, and the results are submitted, the operator is notified whether the drawn crude oil is suitable for use. In the past, the sample results would be used for years, even though the crude qualities might change over time and the mixing time was no longer relevant.

Thus, existing methods of testing crude oil samples can be flawed, due to handling procedures for the sample. Samples sit in the container for a significant amount of time at the site before testing or must be transported to an offsite location for testing. As such, there is a time delay from the initial drawing of crude oil to sending the sample to a testing center, and subsequently in receiving the results of the testing. The timing of receiving results can vary depending on the availability of the testing center. As a result, this method of testing is unpredictable. During the time delay, the operators must choose to either stand idle or risk drawing further potentially impure crude oil. Neither scenario is desirable. In addition to the time cost in sending crude oil samples to an offsite testing facility, there is additional monetary and labor costs associated with such offsite testing. Thus, there is a need for an alternative system for testing crude oil samples that can be completed in a more time efficient manner and that reduces labor and monetary costs.

### SUMMARY OF THE INVENTION

The present invention is directed toward a system for quickly and accurately determining the water cut of a crude oil sample. This invention will reduce or eliminate guessing and averaging and preferably provide correct readings on the site for each individual batch. The system generally includes a crude oil sample container with a circulation loop. A water cut meter may be positioned within the circulation loop, downstream a sample withdrawal inlet and upstream of a sample withdrawal outlet. The water cut meter may be used to provide a near instantaneous measurement of the water cut as the sample of crude oil passes through the circulation loop of the system. This improves upon the process of collecting a sample, allowing the sample to stratify, and sending the sample to a laboratory or testing center to determine the water cut. As one option, this feature will allow the operator to make a quick on-site decision whether to accept the delivered product or to reject it and return to the sender. Previously, such a decision would carry a great degree of uncertainty, since the integrity of the sample aliquot would have been in question. The on-site water cut meter in the sampling system circulation loop provides a much higher degree of accuracy at the site than previously available.

As a second option, a water cut meter incorporated into the circulation loop may further provide an initial reading that can be compared to additional or further testing at a laboratory or testing center. Additionally, if the mixing loop is proven to be stable and mixed, a sub-sample may be drawn off from the collected crude oil by a draw-off valve located downstream of the water cut meter for additional analysis. Thus, as well as providing the water cut of the drawn sample on-site, the water cut meter may further be used to determine whether the sub-sample has stabilized when the sub-sample is drawn off. The sub-sample can then be substantiated and graphed at the testing center, and the results may then be transmitted back to the system operator. With the improved accuracy and on-site readings, custody transfer requirements could be more readily available to both the producing party and the purchasing party.

In any case, the water cut meter may further include a water cut dial with visual indicators to provide the operator with an indication of the purity and stability of the drawn crude oil sample. In one embodiment, the water cut meter may provide a numerical water cut percentage of the drawn crude oil sample. In another embodiment, the water cut dial may comprise a color-coded system that can alert the operator as to the purity or stability of the sample or sub-sample of the drawn crude oil. Further, the present invention may be used in several different crude oil sampling systems that are currently known, or that have yet to be discovered. By way of non-limiting example, the present invention may be used with a condensate sampler, a mixing skid, or a stationary crude container. In any embodiment, the present invention may be used to provide a near instantaneous reading of the water cut of a drawn sample of crude oil or other fluid with detectable water content.

### BRIEF DESCRIPTION OF THE SEVERAL VIEWS OF THE DRAWING

In the accompanying drawings, which form a part of the specification and are to be read in conjunction therewith in which like reference numerals are used to indicate like or similar parts in the various views:

FIG. 1 is a schematic view of a condensate/light crude container with an integrated water cut meter used with a condensate sampler in accordance with a first embodiment of the present invention;

FIG. 2 is a schematic view of a crude container with an integrated water cut meter used with a mixing skid in accordance with a second embodiment of the present invention; and

FIG. 3 is a schematic view of a weathered crude container with an integrated water cut meter used with a stationary crude container in accordance with a third embodiment of the present invention.

### DETAILED DESCRIPTION OF THE INVENTION

The following detailed description of the invention references specific embodiments in which the invention can be practiced. The embodiments are intended to describe aspects of the invention in sufficient detail to enable those skilled in the art to practice the invention. Other embodiments can be utilized, and changes can be made without departing from the scope of the present invention. The present invention is defined by the appended claims and the description is, therefore, not to be taken in a limiting sense and shall not limit the scope of equivalents to which such claims are entitled.

The present invention is generally directed to a system for the testing of crude oil or other liquids, namely, by incorporating a water cut meter into the circulation loop of a crude oil sampler container. The present invention may utilize any currently known water cut meters or water cut meters that have yet to be designed, manufactured, or may otherwise become available in the future. Accordingly, the present invention may further utilize water cut meters of varying sizes, depending on the embodiment.

The present invention may further be used with a variety of models of crude containers and may be adapted for use with any currently known crude containers or crude containers that have yet to be manufactured, discovered, or otherwise become available. In accordance with the present invention, any currently known, or yet to be discovered crude oil containers, extractors, samplers, or other similar devices may be used and are within the scope of the present invention.

In one embodiment, a water cut meter may be positioned or otherwise incorporated into the circulation loop of a crude oil sampler and particularly be positioned downstream of a sample withdrawal inlet to nearly instantaneously measure the water cut of the batch of crude oil being sampled.

In addition to providing an initial water cut of the drawn sample, the present invention may also be used to provide a sub-sample for additional and optional testing of the drawn crude oil. In one embodiment, the present invention may be used to confirm that the sub-sample drawn for the sub-sample is stabilized, and further, the water cut is giving a stable reading at the time the sub-sample is drawn. If stabilized, the sub-sample may then be transported or otherwise sent to a laboratory or testing center for additional or further testing. The additional or further testing at the laboratory or testing center may then be substantiated and graphed remotely at the time that the sub-sample is drawn off to check and back up that the operator at the stationary crude container has allowed enough time and that the water cut meter has shown a stabilized content of water in the circulation loop of the stationary crude container when the sub-sample is withdrawn.

Depending on the initial assessment, the operator may be alerted that the crude oil is at an acceptable purity level and can continue to draw and process the crude oil. Alternatively, the operator may be alerted that the crude oil is at an unacceptable purity level, in which case the drawing process can be discontinued for that location. The near instantaneous analysis of the drawn crude oil provides a time saving benefit to the operator, as opposed to collecting the sample and sending the sample to a laboratory or testing center and waiting for results. Further, the initial analysis may provide a benchmark, should the operator wish to submit the sample or a sub-sample to a laboratory or testing center for secondary verification or further testing.

The invention will now be described with reference to the figures, in which like reference numerals refer to like parts throughout. For purposes of clarity in illustrating the characteristics of the present invention, proportional relationships of the elements have not necessarily been maintained in the figures. It will be appreciated that any dimensions included in the figures are simply provided as examples; and dimensions other than those provided therein are also within the scope of the invention.

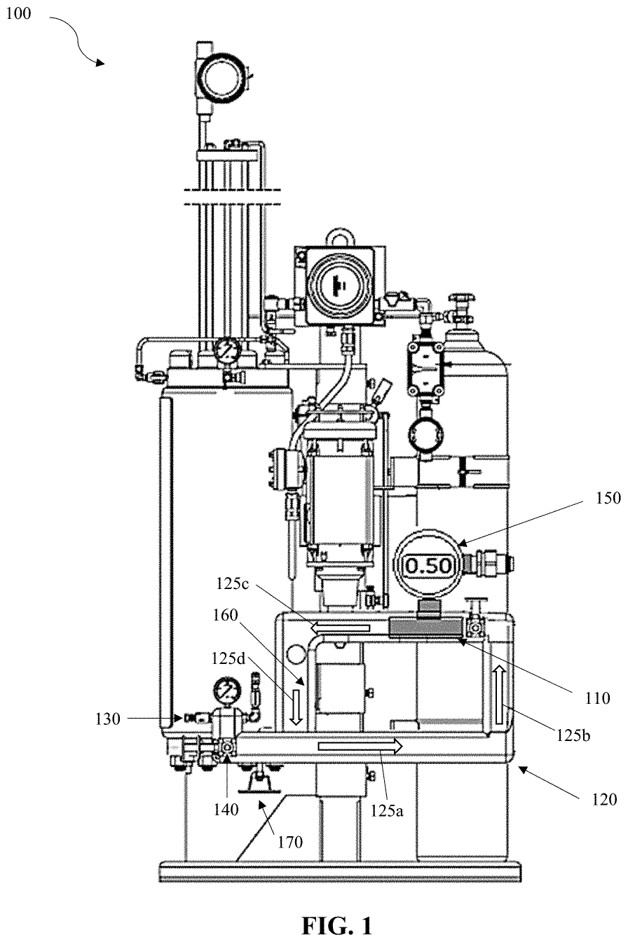

In a first embodiment, the present invention may be compatible with a condensate sampler. By way of non-limiting example, and demonstrated in FIG. 1, the present invention may be used with a condensate sampler 100 such as the WELKER® LS-14 Condensate Sampler. The condensate sampler 100 can be used in a composite sampling system to collect and retain samples of crude oil or other liquids for analysis using a constant pressure sample container to help ensure that the collected sample is maintained in pipeline condition. The condensate sampler 100 may operate at a constant pressure higher than atmospheric pressure. As demonstrated, a water cut meter 110 may be positioned in a circulation loop 120 of the condensate sampler 100 and positioned downstream of a sample withdrawal inlet 130.

As illustrated, arrows 125a-125d show the flow of the sample through the circulation loop 120. As shown by arrow 125a, the sample of crude oil or other liquid enters the circulation loop 120 through the sample withdrawal inlet 130. The sample passes through the circulation loop 120, as shown by arrow 125b, and then enters the water cut meter 110. The water cut meter 110 may be used to measure the water cut value of the sample. The water cut meter 110 may include a water cut dial 150. The water cut dial 150 may include a display screen, or other visual display to provide information to the operator, such as the water cut percentage, volume, flow rate, or other information. To determine when the sample has stabilized, the water cut meter 110 may be programmed to determine that the water cut percentage has remained within a predetermined range for a predetermined period of time.

In one embodiment, the water cut meter 110 may be programmed or otherwise capable of calculating a water cut percent range. When the water cut percent reaches a certain threshold water cut value, the water cut dial 150 may change in color or provide other visual indicators. As such, the water cut meter 110 may be programmed to constantly recalculate the water cut percentage. For example, the water cut dial 150 may start as being red in color, indicating that the water cut percentage is at a relatively high percentage of water. As the collected sample starts to stabilize and decline in water percentage, the water cut dial 150 may become yellow in color to display that the sample has started stabilizing and is declining in water percentage. When the sample has reached a predetermined, desired stabilization, the water cut dial 150 may further change in color to green, indicating to the operator that the sample is stabilized and ready for the operator to extract the sample into a container or storage unit that can then be transported to a laboratory or testing center. As demonstrated, the water cut dial 150 may be attached to the water cut meter 110, however, it will be appreciated that the water cut dial 150 may be positioned in other locations.

After the sample passes through the water cut meter 110a, the sample continues to flow through the circulation loop 120, as shown by arrows 125c, 125d. A portion of the crude oil sample may be removed from the circulation loop 120 through a draw-off valve 140. The draw-off valve 140 may be located at the end of the circulation loop 120. However, it will be appreciated that the draw-off valve 140 may be positioned in other locations. For instance, as shown in FIG. 1, the draw-off valve 140 may be positioned downstream of the sample withdrawal inlet 130 and upstream of the water cut meter 110. The removed portion may be used as a sub-sample and may be transported to a testing center for further and additional testing or may alternatively be used to confirm the initial reading of the water cut meter 110. After passing through the water cut meter 110, the crude oil sample may exit the circulation loop 120 through a sample withdrawal outlet 160 and return the crude oil sample to the condensate sampler 100. The sample withdrawal outlet 160 is positioned downstream of the water cut meter 110.

The water cut meter 110 and/or the water cut dial 150 may provide communication between a remote location monitoring the operation site and the drawing site. A transmitter may direct the analytical information from the water cut meter 110 to a human machine interface located in an operator room or to another third party, such as a foreman, manager, executive, or other third party, such that the third party can see and interpret the analysis of the water cut of the crude oil sample. As such, the third party may be able to verify water cut values determined in the field and at the laboratory. Alternatively, the analysis may be completed automatically using a processor.

The circulation loop 120 may comprise a first isolation valve 170 downstream of the sample withdrawal inlet 130 and upstream of the water cut meter 110. When the first isolation valve 170 is closed, the sample is blocked from flowing through the circulation loop 120 and the water cut meter 110. In some embodiments, the circulation loop 120 may comprise a second isolation valve (not illustrated) downstream of the water cut meter 110 and upstream of the sample withdrawal outlet 160. When the second isolation valve is closed, the sample is prevented from flowing out of the circulation loop 120 through the withdrawal outlet 160. When both the first isolation valve 170 and the second isolation valve are closed, the water cut meter 110 is isolated from the remainder of the circulation loop 120.

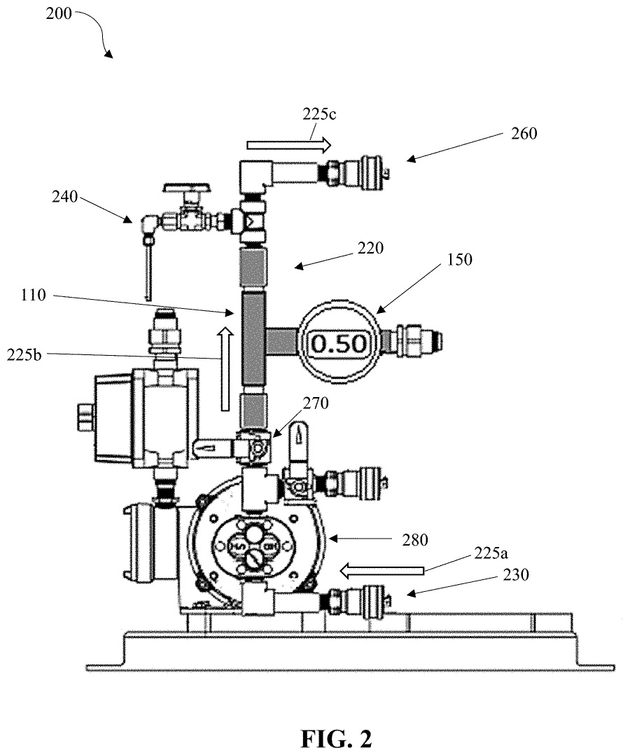

In a second embodiment, the present invention may be compatible with a mixing skid. By way of non-limiting example and as demonstrated in FIG. 2, the present invention may be used with for example a WELKER® MSTCC mixing skid 200. The mixing skid 200 can be used as a stationary closed loop mixing system and can disperse and disburse stratified composite samples for analysis and testing. Similar to the water cut meter 110 of FIG. 1, the water cut meter 110 may be positioned in a circulation loop 220 of the mixing skid 200.

As shown in FIG. 2, the water cut meter 110 may be positioned downstream of a sample withdrawal inlet 230 of the circulation loop 220 but upstream of a draw-off valve 240 of the circulation loop 220. A pump 280 positioned downstream of the sample withdrawal inlet 230 moves the sample through the circulation loop 220. The mixing skid 200 may operate at a constant pressure equal to or greater than atmospheric pressure. As the sample of crude oil or other liquid passes through the circulation loop 220, as illustrated by arrows 225a-225c, the water cut meter 110 may be used to measure the water cut of the sample. Like the water cut meter 110 of FIG. 1 described above, an indication of the water cut value may be displayed on a water cut dial 150 using numerical, color coding, or other indicators of purity. As demonstrated, the water cut dial 150 may be attached to the water cut meter 110, however, it will be appreciated that the water cut dial 150 may be positioned in other locations.

After passing through the circulation loop 220 and after the initial water cut can be displayed on the water cut dial 150, a portion of the sample may be removed from the circulation loop 220 through the draw-off valve 240 located near the end of the circulation loop 220. The removed portion may be used as a sub-sample and may be transported to a testing center for further and additional testing or may alternatively be used to confirm the initial reading of the water cut meter 110. Like the circulation loop 120 of FIG. 1, the circulation loop 220 may have a first isolation valve 270 positioned downstream of the sample withdrawal inlet 230 and upstream of the water cut meter 110. When the first isolation valve 270 is closed, the sample is blocked from flowing through the circulation loop 220 and the water cut meter 110. In some embodiments, the circulation loop 220 may comprise a second isolation valve (not illustrated) downstream of the water cut meter 110 and upstream of the sample withdrawal outlet 260. When the second isolation valve is closed, the sample is prevented from flowing out of the circulation loop 220 through the sample withdrawal outlet 260. This feature is only for maintenance of the water cut meter during which time the system must be turned off. When both the first isolation valve 270 and the second isolation valve are closed, the water cut meter 110 is isolated from the remainder of the circulation loop 220.

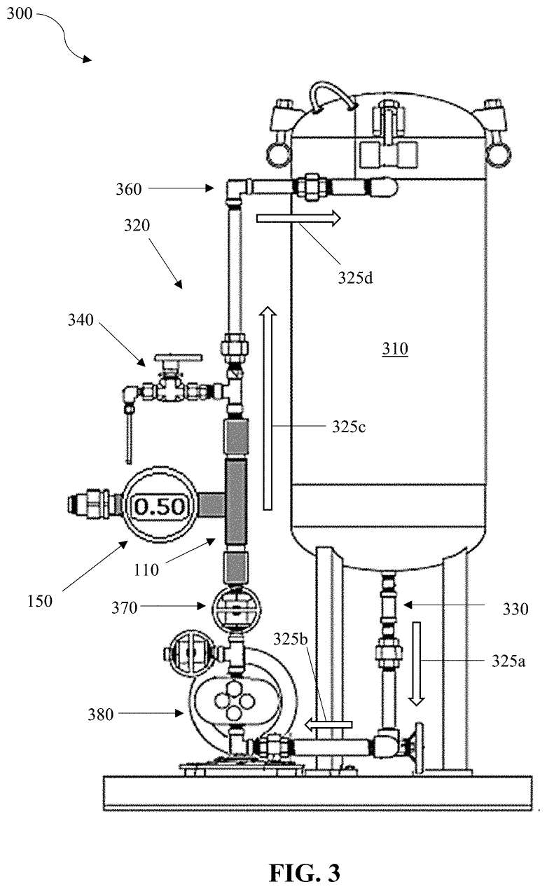

In a third embodiment, the present invention may be compatible with a stationary crude container. By way of non-limiting example and as demonstrated in FIG. 3, the present invention may be used with for example a WELKER® SCC (stationary crude container) 300. The stationary crude container 300 can be used as a stationary closed loop mixing system and is particularly well suited for the mixing, sampling, and testing of large batches of crude oil or other liquids. Similar to the described embodiments of FIGS. 1 and 2, the water cut meter 110 may be positioned in a circulation loop 320 of the crude container 300.

As illustrated, the water cut meter 110 may be positioned in a circulation loop 320. Similar to the circulation loop 120 of FIG. 1, and the circulation loop 220 of FIG. 2, the water cut meter 110 may be positioned downstream of a storage tank 310, a sample withdrawal inlet 330, and a pump 380. The stationary crude container 300 may operate at atmospheric pressure or at a pressure higher than atmospheric pressure. As further demonstrated in FIG. 3, the water cut meter 110 may be positioned upstream of a draw-off valve 340 located near the end of the circulation loop 320.

As the sample of crude oil or other liquid passes through the circulation loop 320, as illustrated by arrows 325a-325d, the water cut meter 110 may be used to measure the water cut of the sample. As discussed above, the water cut may be displayed on the water cut dial 150 using numerical, color coding, or other indicators of purity. As shown, the water cut dial 150 may be attached to the water cut meter 110, however, it will be appreciated that the water cut dial 150 may be positioned in other locations. The removed portion may be used as a sub-sample and may be transported to a testing center for further and additional testing or may alternatively be used to confirm the initial reading of the water cut meter 110. After passing through the water cut meter 110, the crude oil sample is returned to the storage tank 310 through a sample withdrawal outlet 360 positioned between the water cut meter 110 and the storage tank 310.

Like the circulation loop 120 of FIG. 1, and the circulation loop 220 of FIG. 2, the circulation loop 320 may have a first isolation valve 370 positioned downstream of the sample withdrawal inlet 330 and upstream of the water cut meter 110. When the first isolation valve 370 is closed, the sample is blocked from flowing through the circulation loop 320 through water cut meter 110. In some embodiments, the circulation loop 320 may comprise a second isolation valve (not illustrated) downstream of the water cut meter 110 and upstream of the sample withdrawal outlet 360. When the second isolation valve is closed, the sample is prevented from flowing out of the circulation loop 320 and back into the storage tank 310. When both the first isolation valve 370 and the second isolation valve are closed, the water cut meter 110 is isolated from the remainder of the circulation loop 320. This feature is only for maintenance of the water cut meter during which time the system must be turned off.

As described herein, the three embodiments do not presently incorporate a static mixer in the system of drawing, sampling, and testing of the drawn crude oil. Instead, the three described embodiments may utilize a circulation pump, an electrically driven pump, and a pneumatically driven pump to provide an effective mix of water for sampling from the drawn crude oil. However, in alternate embodiments a static mixer can be implemented and used in the system. For example, a static mixer may be positioned between the sample withdrawal inlet 130 and the water cut meter 110 of the circulation loop 120 of FIG. 1, or between the sample withdrawal inlet 230 and the water cut meter 110 of the circulation loop 220 of FIG. 2. Additionally, as further shown in FIGS. 2 and 3, the draw-off valve 240 and 340, respectively, may be located directly downstream and substantially aligned with the water cut meter 110 in a straight run of pipe, as opposed to being located in an elbow joint located with the draw-off valve which could be in a problematic position as related to the profile of the drawn water, due to the water turning a corner and “barber poling” the flow of water. This problem is particularly present in older models of crude oil samplers, and thus, positioning the water cut meter in a straight turn of pipe allows the present invention to be combined with both newer and older crude oil samplers. Each of the three embodiments may be stationary skids designed to be permanently affixed to a location. Alternatively, each of the three embodiments may be transportable skids configured to be transportable from a first location to a second location.

From the foregoing, it will be seen that this invention is one well adapted to attain all the ends and objects hereinabove set forth together with other advantages which are inherent to the structure and method. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. Thus, there has been shown and described several embodiments of a novel invention. As is evident from the foregoing description, certain aspects of the present invention are not limited by the particular details of the examples illustrated herein, and it is, therefore, contemplated that other modifications and applications, or equivalents thereof, will occur to those skilled in the art. The terms “having” and “including”, and similar terms as used in the foregoing specification are used in the sense of “optional” or “may include” and not as “required”. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A system for the testing and sampling crude oil, the system comprising:
a circulation loop comprising;
a sample withdrawal inlet into which a crude oil sample may enter the circulation loop;
a water cut meter downstream of the sample withdrawal inlet configured to:
determine a water cut value of the crude oil sample;
compare the water cut value to a threshold water cut value;
a draw-off valve downstream of the water cut meter configured to permit a sub-sample of the crude oil sample to be removed from the circulation loop; and
a sample withdrawal outlet downstream of the draw-off valve through which the crude oil sample exits the circulation loop.

2. The system of claim 1, wherein the circulation loop comprises a water cut dial connected to the water cut meter for visually displaying the water cut value.

3. The system of claim 1, wherein the circulation loop comprises a static mixer positioned between the sample withdrawal inlet and the water cut meter for mixing the crude oil sample prior to the crude oil sample entering the water cut meter.

4. The system of claim 1, wherein the water cut meter comprises a transmitter configured to communicate the water cut value to a monitoring system.

5. The system of claim 1, wherein after the water cut meter compares the water cut value to a threshold value, the water cut meter is configured to continuously recalculate the water cut value at a predetermined interval of time.

6. The system of claim 1, wherein the circulation loop comprises a straight run of pipe positioned between the sample withdrawal inlet and the sample withdrawal outlet ; and wherein the water cut meter and the draw-off valve are positioned along the straight run of pipe.

7. The system of claim 1, wherein the circulation loop comprises:
a first isolation valve upstream of the water cut meter and downstream of the sample withdrawal inlet;
a second isolation valve downstream of the water cut meter and upstream of the sample withdrawal outlet;
wherein, the first isolation valve is for selectively blocking the crude oil sample from entering the water cut meter; and
wherein, the second isolation valve is for selectively blocking the crude oil sample from exiting the circulation loop during maintenance only.

8. The system of claim 7, wherein when the first isolation valve is closed, the crude oil sample is blocked from entering the water cut meter.

9. The system of claim 7, wherein when the second isolation valve is closed, the crude oil sample is blocked from entering the sample withdrawal outlet.

10. The system of claim 7, wherein when both of the first isolation valve and the second isolation valve are closed, the water cut meter is isolated from a remainder of the circulation loop.

11. A system for the testing and sampling crude oil, the system comprising:
a skid comprising:
a sample container configured to contain a crude oil sample; and
a circulation loop connected to the sample container, the circulation loop comprising;
a water cut meter configured to determine a water cut of the crude oil sample;
a sample withdrawal inlet positioned downstream of the sample container and upstream of the water cut meter configured to permit the crude oil sample to flow from the sample container through the water cut meter;
a water cut dial coupled to the water cut meter configured to visually display the water cut value;
a draw-off valve positioned downstream and substantially aligned with the water cut meter; and
a sample withdrawal outlet positioned downstream of the draw-off valve and upstream of the sample container configured to return the crude oil sample to the sample container.

12. The system of claim 11, wherein the skid is a stationary skid is affixed to a location.

13. The system of claim 11, wherein the skid is a temporary skid configured to be transportable from a first location to a second location.

14. The system of claim 11, wherein the circulation loop operates at atmospheric pressure.

15. The system of claim 11, wherein when the skid is a condensate sampler, the circulation loop operates at a pressure higher than atmospheric pressure.

16. The system of claim 11, wherein when the sample container of the skid is connected to a primary circulation loop, the circulation loop is auxiliary to the primary circulation loop.

17. The system of claim 11, wherein the skid comprises an electrically driven pump downstream of the sample withdrawal inlet and upstream of the water cut meter; and wherein the electrically driven pump is configured to mix the crude oil sample prior to entering the water cut meter.

18. The system of claim 11, wherein the skid comprises a pneumatically driven pump downstream of the sample withdrawal inlet and upstream of the water cut meter; and wherein the pneumatically driven pump is configured to mix the crude oil sample prior to entering the water cut meter.

19. The system of claim 11, wherein the skid comprises a circulation pump downstream of the sample withdrawal inlet and upstream of the water cut meter; and wherein the circulation pump is configured to mix the crude oil sample prior to entering the water cut meter.

20. The system of claim 11, wherein the system is a closed loop system.
